(19) United States
(12) Patent Application Publication
(10) Pub. No.: US 2013/0190672 A1
(43) Pub. Date: Jul. 25, 2013

(54) NONADHERENT AND SUPERABSORBENT WOUND DRESSINGS BASED ON ELECTROSPUN ZWITTERIONIC MONOMERS

(71) Applicant: The University of Akron, Akron, OH (US)

(72) Inventor: Lingyun Liu, Tallmadge, OH (US)

(73) Assignee: The University of Akron, Akron, OH (US)

(21) Appl. No.: 13/749,913

(22) Filed: Jan. 25, 2013

Related U.S. Application Data
(60) Provisional application No. 61/590,457, filed on Jan. 25, 2012.

(51) Int. Cl. A61L 15/24

(52) U.S. Cl. CPC A61L 15/24
USPC 602/45; 526/288; 264/477

(57) ABSTRACT

A method for creating a non-adhesive, water-absorbent wound dressing includes creating a mixture of monomers, crosslinker, initiator, and solvent, initiating polymerization of the monomers to begin polymerization of the monomers; quenching the polymerization of the monomers when the mixture has a viscosity suitable for electrospinning; electrospinning the polymer mixture to form a fibrous membrane; and thereafter further polymerizing and crosslinking the fibrous membrane. The monomers are chosen from select species and, following this process, provide wound dressings that are nonadherent to a wound so as to be potentially reusable; superabsorbent; non-biofouling. With the addition of antimicrobial cations, the wound dressings also provide antimicrobial properties.

### CROSS-REFERENCE TO RELATED APPLICATION

This application is a non-provisional of U.S. Patent Provisional Application No. 61/590,457, filed on Jan. 25, 2012, the contents of which are incorporated herein by reference.

### FIELD OF THE INVENTION

The present invention relates to wound dressings. In particular embodiments, the present invention relates to nonadherent and superabsorbent wound dressings based on electrospun zwitterionic monomers and to a method of making the same.

### BACKGROUND OF THE INVENTION

Wound dressings are generally used to cover wounds in an effort to assist in the wound healing process. Wound dressings play a major role in wound care, especially to treat thermal, traumatic, and chronic wounds. Covering wounds with dressings has been shown to increase the rate of epithelialization, heal wounds faster, and effectively manage wound infection. An ideal wound dressing will protect the wound from microorganism infection, allow gas exchange, absorb exudate, impart a moist environment to enhance epithelial regrowth and be painless to remove. Cotton gauze has traditionally been used in wound dressings. However, it allows fast evaporation of fluids and makes the wound desiccate. The porous structure also does not provide an efficient barrier against bacterial penetration. As a consequence, the wound dressing may adhere to the wound bed and cause severe pain and bleeding upon removal.

A wide variety of materials can be used for wound dressings. Typical natural materials used include chitosan, chitin, alginate, cellulose acetate, cellulose, hyaluronic acid, collagen, silk, and gelatin. Limitations associated with the use of natural materials include batch-to-batch variations of raw materials and the possibility of disease transmission. Recently, natural biomaterials, synthetic polymers, or their blends have been explored for wound dressing applications in various forms such as film, hydrogel, foam, or fiber.

Synthetic polymers such as polyurethane, poly(L-lactide), poly(c-caprolactone), polyacrylonitrile, poly(acrylamide)/poly(vinyl sulfonic acid sodium salt), and poly (vinyl alcohol) have also been used as materials for wound dressings. Synthetic polymers offer strong mechanical properties, more reliable lot-to-lot uniformity, and lower cost. Among these materials, few can effectively resist nonspecific protein adsorption from real-world complex media and prevent cell attachment and bacterial adhesion. Biofouling properties of these materials will cause bacterial accumulation on dressing surfaces and cause pain and/or wound aggravation upon dressing removal. In addition, most of the synthetic materials used are either hydrophobic or slightly hydrophilic. They cannot effectively handle excessive wound exudates which promote bacterial growth.

Thus, a need in the art exists for an improved wound dressing that provides one or more and preferably multiple of the following properties: nonadherent to a wound so as to be potentially reusable; superabsorbent; non-biofouling; and antimicrobial.

### SUMMARY OF THE INVENTION

In one or more embodiments the present invention provides a non-adhesive, water-absorbent wound dressing comprised of polymerized monomers according to the following structure:

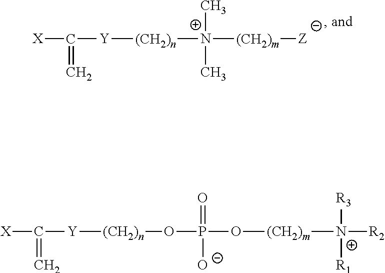

wherein X is selected from the group consisting of hydrogen or a methyl group; Y is selected from the group consisting of esters and amides; n is an integer from 1 to 4, m is an integer from 1 to 5, Z is selected from the group consisting of SO₃ or COO; and R₁, R₂ and R₃ are each any moiety.

In one or more embodiments the present invention provides a method of creating a non-adhesive, water-absorbent wound dressing comprising: creating a mixture of monomers, crosslinker, initiator, and solvent, the monomer being selected from the following structures:

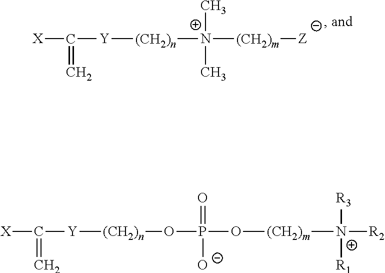

wherein X is selected from the group consisting of hydrogen or a methyl group; Y is selected from the group consisting of esters and amides; n is an integer from 1 to 4, m is an integer from 1 to 5, Z is selected from the group consisting of SO₃ or COO; and R₁, R₂ and R₃ are each any moiety; initiating polymerization of the monomers to begin polymerization of the monomers; quenching the polymerization of the monomers when the mixture has a viscosity suitable for electrospinning; electrospinning the polymer mixture to form a fibrous membrane; and thereafter crosslinking the fibrous membrane.

### BRIEF DESCRIPTION OF THE DRAWINGS

Advantages of the present invention will become better understood with regard to the following description, appended claims, and accompanying drawings wherein:

FIG. 1 is a chart of the water absorption of electrospun (ES) PSBMA membrane according to this invention, as compared to PSBMA hydrogel (HG).

FIG. 2 is a chart of human plasma fibrinogen (Fg) adsorption on electrospun PSBMA membranes according to this invention measured by enzyme-linked immunosorbent assay (ELISA), with electrospun PSBMA membranes with no exposure of Fg and anti-Fg during ELISA being used as negative controls and electrospun polycaprolactone (CL) membrane used as the positive control, and with relative adsorption values (mean ±standard deviation %) shown on top of the columns.

FIG. 3 is a chart of bovine aortic endothelial cell (BAEC) attachment on the electrospun PSBMA membranes of this invention as assessed by MTT assay after culturing for 48, 72, and 96 hours. Electrospun PCL membrane and TCPS surface were used as the positive controls. Electrospun PSBMA membranes cultured with medium (no cell exposure), PSBMA hydrogel, and PSBMA hydrogel cultured with medium (no cell exposure) were used as the negative controls.

### DETAILED DESCRIPTION OF ILLUSTRATIVE EMBODIMENTS

The present invention relates to nonadherent and superabsorbent wound dressings based on monomers according to the following structures:

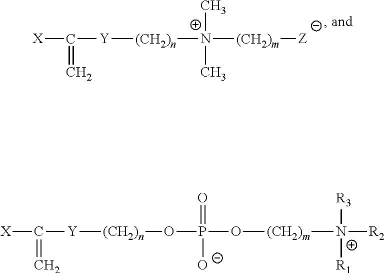

wherein X is selected from the group consisting of hydrogen or a methyl group; Y is selected from the group consisting of an esters and amides; n is an integer from 1 to 4, m is an integer from 1 to 5, Z is selected from the group consisting of SO₃ or COO; and R₁, R₂ and R₃ are each any moiety. In other embodiments, R1, R2 and R₃ are small alkyl groups (of from C1 to C3, i.e. methyl, ethyl or propyl groups).

In particular embodiments employing the first structure, X is methyl, Y is ester, n is 2, m is 3, and Z is SO₃, such that the monomer is sulfobetaine methacrylate (SBMA). In other embodiments, X is methyl, Y is ester, n is 2, m is 2, and Z is COO, such that the monomer is carboxybetaine methacrylate (CBMA). In still other embodiments, X is hydrogen, Y is amide, n is 2, m is 1, 3, or 5, and Z is COO, such that the monomer is carboxybetaine acrylamide (CBAA).

In particular embodiments employing the second structure, X is methyl, Y is ester, n is 2, m is 2 and R₁, R₂ and R₃ are each a methyl group, such that the monomer is poly(phosphoryl)choline.

In accordance with this invention, the monomers as described above are polymerized, electrospun, and crosslinked to form fibrous membranes that can serve as wound dressings. In some embodiments, a single species of monomer is employed such that a homopolymer is created. In other embodiments, more than one species of monomer is employed such that a copolymer is created.

The polymers formed are represented by the following structures:

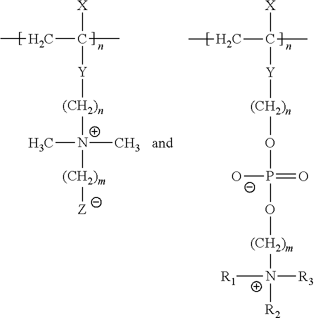

wherein X is selected from the group consisting of hydrogen or a methyl group; Y is selected from the group consisting of an esters and amides; n is an integer from 1 to 4, m is an integer from 1 to 5, Z is selected from the group consisting of SO₃ or COO; and R₁, R₂ and R₃ are each any moiety. In other embodiments, R1, R2 and R₃ are small alkyl groups (of from C1 to C3, i.e. methyl, ethyl or propyl groups).

These polymers are electrospun to form fibrous membranes (non-wovens) that are then crosslinked to form a wound dressing. The crosslinked fibrous membranes are water stable. Notably hydrogel formulation and experimental conditions cannot be directly applied to the electrospinning process since electrospinning requires a viscous solution with a relatively consistent viscosity throughout the process. In order to successfully electrospin the polymers taught herein and form useful wound dressing, a special process must be followed. This is because, upon polymerization, there is a rapid increase in the reactant solution viscosity prior to the gel formation, meaning that there is only a very short time window where the solution can be electrospun. Once hydrogel is formed, conversion of such hydrogel into fibrous form (i.e., electrospinning) is impossible since the cross-linked bulk gel can neither dissolve nor melt.

Thus, the present invention provides a three step polymerization-electrospinning-photocrosslinking process to fabricate the water-stable crosslinked electrospun membrane. A mixture of the monomer, crosslinker, redox initiators, and photoinitiator is first reacted via free radical polymerization to form a precursor solution to achieve a viscosity necessary for electrospinning. The reaction is then cold-quenched to stop further increase in solution viscosity. Next, the viscous precursor solution is electrospun at a low temperature. The actions of quenching the polymer solution and keeping the electrospinning solution at a low temperature both assure a relatively consistent solution viscosity during the electrospinning and prevent the gel formation which otherwise would clog the spinning tip of the electrospinning apparatus. Upon completion of electrospinning, the membranes are white, pliable and stretchable. Further polymerization and crosslinking is carried out after the electrospinning to complete the wound dressing membrane. By quenching the polymerization (i.e., effecting incomplete polymerization), double bonds are available from the unreacted monomer molecules or on the polymer chains, and can be utilized for further polymerization. In some embodiments, the addition of TEGDMA further enhances the availability of double bonds, leading to the crosslinked PSBMA with the aid of the UV initiator, Irgacure 2959. Before the UV treatment, the electrospun membranes dissolved readily in water.

First, the chosen monomer or monomers are polymerized in a solvent to achieve a viscosity suitable for electrospinning The monomer(s) are mixed with appropriate initiators, appropriate crosslinker, and appropriate solvent, and polymerization is then initiated and subsequently controlled to facilitate a subsequent electrospinning step. Those of ordinary skill in the art will be able to determine suitable initiators, crosslinkers and solvents based on the desired procedure for polymerization and crosslinking, and, similarly, one of ordinary skill in the art will be able to determine amounts of each component to carry out the method and practice the wound dressings of this invention.

Suitable initiators will be known and readily determined by those of ordinary skill in the art. In some embodiments, redox initiators are employed to initiate polymerization prior to electrospinning, and are selected from the group consisting of sodium metabisulfate (SBS), ammonium persulfate (APS). A photoinitiator such as 2-hydroxy-1-[4-(2-hydroxyethoxy)phenyl]-2-methyl-1 -propanonecan also be used to reinitiate polymerization and effect some degree of crosslinking after electrospinning.

Suitable crosslinkers will be known and readily determined by those of ordinary skill in the art. In some embodiments, the crosslinker is a crosslinker that is activated upon being subjected to a particular temperature. An example of a suitable thermal initiator is 2,2′-Azobis(2-methylpropionitrile). It is noted that the 2-hydroxy-1-[4-(2-hydroxyethoxy)phenyl]-2-methyl-1-propanone photoinitiator will resume the polymerization and crosslinking during the final formation step, after electrospinning of the polymer(s) forming the wound dressing. A particular crosslinker used here is TEGDMA (tetraethylene glycol dimethacrylate). Any other crosslinkers that contain two double C═C bonds could serve as the crosslinker, e.g., di(ethylene glycol) dimethacrylate, ethylene glycol dimethacrylate, and tetra(ethylene glycol) diacrylate

Suitable solvents will be known and readily determined by those of ordinary skill in the art. In some embodiments, the solvent is selected from the group consisting of water, methanol, and ethanol and mixtures thereof.

After creating the polymerizable mixture, the polymerization is initiated and carried out to achieve a desired viscosity suitable for electrospinning. Various means can be used for initiating the polymerization depending on the initiator that is used. Suitable initiators will be known and readily determined by those of ordinary skill in the art.

When redox initiators are employed, the polymerizable mixture begins to polymerize upon mixing of the components. When a thermal initiator is employed, the polymerizable mixture is subjected to heat to begin polymerization. When a photoinitiator is used, the polymerizable mixture is subjected to any light to begin polymerization. When a UV initiator is used, the polymerizable mixture is subjected to UV light to begin polymerization. Other initiators and methods of initiation are generally known in the art.

The viscosity suitable for electrospinning is determined in a very general manner known in the art. The viscosity of the polymerizable mixture increases as polymerization progresses, and those of ordinary skill in the art can determine suitable viscosities of electrospinning without undue experimentation. The polymerizable mixture merely needs to be thick enough so as to hold up under the electrospinning process but not so thick that it clogs the spinning tip or cannot be drawn into fibers through the electrospinning process. This can actually be determined by simple observation. As the less viscous mixture becomes more viscous upon polymerization, the effect can be observed by simple manipulation of the mixture (as by tilting a beaker containing the polymerizing mixture), and the electrospinning can be carried out when the viscosity is deemed appropriate. If incorrect, it is a minor detail to run the experiment again and stop the polymerization sooner or later depending on the success of the first run.

After polymerizing to a viscosity suitable for electrospinning, the temperature is reduced in order to slow the rate of polymerization and maintain the viscosity at a range suitable for electrospinning. This can be achieved by a simple cooling of the polymerizing mixture. The mixture at the desired viscosity is placed in a cold environment to reduce the temperature of the mixture and halt or at least slow polymerization to stretch out the time during which the polymer solution may be electrospun.

After slowing the polymerization, the mixture is electrospun. Electrospinning is a simple, yet effective method of producing fibrous membranes with high surface-area-to-volume ratio. It provides several attributes important for wound dressings, including high absorptivity due to high surface-area-to-volume ratio, high gas permeation due to the porous structure, and conformability to contour of the wound area.

Electrospinning involves converting the polymerizable mixture into a non-woven fabric of nanofibers. The polymerizable mixture solution is added to the electrospinning apparatus. Therein, the solution is fed into the needle or syringe where it will be extruded from the tip. The extrusion occurs by applying a charge to the needle or syringe, and results in the extrusion of a fiber from the solution onto a nearby grounded plate. As the extrusion continues, a non-woven fabric of the nanofibers develops on the grounded plate. This method of electrospinning and other related methods are well known in the art.

During the electrospinning step, it is important to control the humidity. By controlling the humidity, the evaporation of the solvent can be controlled to leave behind well formed electrospun fibers. In one embodiment, the humidity is maintained at 20% or less. In certain embodiments, the humidity is maintained at 15% or less, in other embodiments, at 10% or less, and, in other embodiments, at 5% or less. In some embodiments, the humidity is maintained at from 0% or more to 20% or less, in other embodiments, from 0% or more to 15% or less, in other embodiments, from 0% or more to 10% or less, in other embodiments, from 0% or more to 5% or less, in other embodiments, from 5% or more to 15% or less and in still other embodiments, from 5% or more to 10% or less.

The temperature is also controlled during the electrospinning step in order to control the rate of polymerization and thereby avoid a viscosity increase that would frustrate the electrospinning process.

In certain embodiments, the temperature is maintained from 0° C. or higher to 20° C. or lower, in other embodiments, from 0° C. or higher to 15° C. or lower, and in still other embodiments, from 5° C. or higher to 15° C. or lower.

As described above, the mixture of the present invention is electrospun to form non-woven fabric that can serve as a wound dressing.

Following the electrospinning step, the mixture is crosslinked according to the type of crosslinking agent employed in the polymerizable mixture. Crosslinking occurs when polymer chains are connected to each other. One example of crosslinking is to employ a UV initiator, such as that mentioned above. When the UV light is applied to the mixture, the crosslinking occurs. Also upon application of the UV light, the polymerization of the mixture is continued to completion or near completion.

The wound dressings of the present invention exhibit beneficial properties over existing wound dressings. The beneficial properties include improved water stability, water absorption, and an increased resistance to protein adsorption, cell adhesion, and bacterial attachment; therefore, the dressing removal will neither cause patients pain nor disturb the newly formed tissues. The dressings also prevent attachment of environmental bacteria and offers broad-spectrum antimicrobial activity.

Some embodiments of the present invention contain zwitterionic monomers. Compounds that are zwitterionic are both ultralow biofouling and superhydrophilic. Zwitterionic compounds contain a 1:1 (or approximate thereto) mixture of positive and negative charges within the compound in order to obtain these properties.

The wound dressings of this invention are beneficially superhydrophilic such that they serve to absorb exudate and maintain a moist environment at the wound site. Previously, a moist wound environment has been created by covering the wound with a polymer dressing that significantly increased the rate of epithelialization. The present invention has the advantage of providing a wound dressing that is superhydrophilic.

Biofouling is the attachment and accumulation of organisms on surfaces, particularly those surfaces that are wet. The wound dressings of the present invention exhibit ultralow biofouling. This characteristic prevents bacteria and other organisms from attaching or adhering to the wound dressing, which keeps the wound area clean.

The electrospun membranes made by this method are stable in water. Previously there were no methods to create water-stable fibers of superhydrophilic materials such as PSBMA by electrospinning or other means. The wound dressings of the present invention are stable in the presence of water, providing the advantage that they do not dissolve in the present of sweat or other bodily fluids.

The wound dressings of the present invention are highly water absorbent. The superior water absorption aids in fluid removal from highly exudating wounds while keeping the wound hydrated to support healing. As will be seen in the example herein, the electrospun membranes in accordance with this invention have exhibit high water absorption of 353% (w/w), whereas the similar hydrogel made by conventional methods only absorbed 81% water.

Protein adsorption is the attachment and accumulation of proteins on a surface. The electrospun membrane showed strong resistance to protein adsorption. When a wound occurs, the body uses proteins to heal and repair the wound. Resistance to protein adsorption is desired so that the proteins stay in the area of the wound instead of attaching to the wound dressing.

Similarly, cell adhesion is the attachment of other cells to a surface. The electrospun membrane also showed strong resistance to cell attachment. Resistance to cell adhesion is again desired so that the cells stay in the area of the wound instead of attaching to the wound dressing.

Embodiments of the present invention also provide wound dressings that have improved resistance to bacterial attachment. This is desired in order to prevent the bacteria from infiltrating the wound area and entering the wound.

Bacterial adhesion studies using Gram-negative P. aeruginosa and Gram-positive S. epidermidis showed that the PSBMA electrospun membrane was highly resistant to bacterial adhesion. The Ag+-impregnated electrospun PSBMA membrane was shown microbicidal, against both S. epidermidis and P. aeruginosa.

The improved resistance to protein adsorption, cell adhesion, and bacterial attachment also gives the wound dressings beneficial practical advantages. Some embodiments of the present invention can be reused on multiple wounds. The improved resistance to protein adsorption, cell adhesion, and bacterial attachment gives wound dressings where the dressing does not stick to the skin and where the proteins and cells do not stick to the dressing. Therefore, the dressing can be reused because it can be removed from the patient and rinsed before reuse.

In a specific embodiment, the wound dressing is made with polymerized sulfobetaine methacrylate monomer, poly(sulfobetaine methacrylate) (PSBMA), which has superhydrophilic and ultralow biofouling properties.

The unique characteristics are attributed to its strong hydration capacity, dictated by electrostatic attractions between charges on the polymer pendant groups and water molecules. PSBMA surfaces are ultralow fouling to adsorption from both single protein solutions and complex media such as human blood serum and plasma. The PSBMA hydrogel is highly resistant to cell adhesion both in vitro and in vivo. PSBMA surfaces also inhibit bacterial adhesion and biofilm formation. Another benefit of PSBMA is that it contains a large amount of anionic SO₃⁻ groups (one SO₃⁻ per pendant group), which allow for the incorporation of antimicrobial cationic silver ions. PSBMA is also non-cytotoxic, biocompatible, and biomimetic.

Being superhydrophilic, PSBMA fibers fabricated by a conventional electrospinning method would readily dissolve in water. Electrospinning provides multiple desirable features for wound dressings, including high absorptivity due to high surface-area-to-volume ratio, high gas permeation, and conformability to contour of the wound bed. A three step polymerization-electrospinning-photocrosslinking process was developed to fabricate the crosslinked electrospun PSBMA fibrous membrane.

In a specific embodiment, the polymerizable mixture uses SBMA as the monomer, sodium metabisulfite (SBS) and ammonium persulfate (APS) as redox initiators, 2-hydroxy-1-[4-(2-hydroxyethoxy)phenyl]-2-methyl-1-propanone as a photoinitiator, and tetraethylene glycol dimethacrylate (TEGDMA) as a crosslinker.

In a specific embodiment, the SBS (0.6%, w/v) and APS (1.6%, w/v) are dissolved in 2.5 ml water. The photoinitiator (3.6%, w/v) is then dissolved separately in 2.5 ml methanol. The initiator/water solution and photoinitiator/methanol solution are mixed homogenously. Then, the crosslinker (92.5 μl) and monomer (1.8861 g) are then added to the overall mixture. This mixture is then polymerized, electrospun, and photocrosslinked.

In other embodiments, the wound dressings are made to have antimicrobial properties by the inclusion of antimicrobial cations that associate with the local negative charge on the mer units resulting from the polymerization of the zwitterionic monomers taught herein.

In accordance with one method, the electrospun wound dressing fabric is immersed in a solution containing antimicrobial cations and the antimicrobial cations associate with the local negatively charged species in the polymer of the fabric. For example, when an electrospun wound dressing fabric made from the broad monomer compound described above is immersed in a silver nitrate solution, the positively-charged silver ions will associate with the negatively-charged Z group of the broad monomer compound.

### EXAMPLES

### Materials:

The monomer, N-(3-sulfopropyl)-N-(methacryloxyethyl)-N,N-dimethylammonium betaine (SBMA, H₂C═C(CH₃)COOCH₂CH₂N(CH₃)₂(CH₂)₃SO₃), and the initiators, sodium metabisulfite (SBS) and ammonium persulfate (APS), were purchased from Sigma-Aldrich (Milwaukee, Wis.) and used as received. The photoinitiator, 2-hydroxy-1-[4-(2-hydroxyethoxy)phenyl]-2-methyl-1-propanone (Irgacure 2959) (4-(2-hydroxyethoxy)phenyl-(2-hydroxy-2-propyl)ketone), was supplied by Ciba Specialty Chemicals (Switzerland). Tetraethylene glycol dimethacrylate (TEGDMA) was purchased from Fluka. Polycaprolactone (PCL, Mn=70,000-90,000) was obtained from Aldrich. Methanol was purchased from VWR (Radnor, Pa.). Ethanol (absolute 200 proof) was acquired from Pharmoco-AAPER. Hydrogen peroxide and o-phenylenediamine (OPD) were obtained from Sigma-Aldrich. Sulfuric acid was purchased from EMD Chemicals (Gibbstown, N.J.). Water used in the experiments was purified to a minimum resistivity of 18.0 MΩ-cm by a Millipore filter system. Phosphate-buffered saline (PBS, pH 7.4, 10 mM, 138 mM NaCl, 2.7 mM KCl) and phosphate-citrate buffer (pH 5.0) were purchased from Sigma-Aldrich. Human plasma fibrinogen (Fg) and bovine serum albumin (BSA) were purchased from Sigma-Aldrich. Horseradish peroxidase (HRP)-conjugated polyclonal goat anti-human fibrinogen was obtained from USBiological (Swampscott, Mass.). Bovine aortic endothelial cells (BAECs) were kindly supplied by Prof. Shaoyi Jiang (University of Washington). All cell culture medium and reagents and Vybrant® MTT cell proliferation assay kit were purchased from Invitrogen (Carlsbad, Calif.). Pseudomonas aeruginosa (P. aeruginosa) PA01 with green fluorescent protein (GFP), expressing plasmid was supplied by Dr. Soren Molin of the Technical University of Denmark. Staphylococcus epidermidis (S. epidermidis) RP62A was purchased from ATCC (Manassas, Va.). Live/Dead BacLight viability kit was obtained from Invitrogen.

### Preparation of the Electrospun PSBMA Membranes:

Redox initiators SBS (0.6%, w/v) and APS (1.6%, w/v) were dissolved in 2.5 ml water. Irgacure 2959 (3.6%, w/v) was dissolved in 2.5 ml methanol. Two solutions were mixed homogenously, followed by the addition of TEGDMA (92.5 μl) and SBMA (1.8861 g). The mixture was then placed in a water bath at 38° C. under static conditions for 40 minutes to produce a viscous solution. Polymerization was then quenched at −20° C. for 5 minutes prior to electrospinning.

The basics of electrospinning are generally known. The whole experimental apparatus was placed in a chamber to control the humidity and to prevent fiber collection from being disturbed by air turbulence. A high-voltage power supply (ES30P-5W, Gamma High Voltage Research, Ormond Beach, Fla.) was used to generate adjustable high DC voltage. The polymer solution to be electrospun was loaded into a 5-mL syringe (BD Biosciences, San Jose, Calif.) and delivered using a syringe pump (SP101i, World Precision Instruments, Sarasota, Fla.). The syringe was connected to fluorinated ethylene propylene tubing ( 1/16″ ID, Saint-Gobain, France). The other end of the tube was connected to a vertically oriented 21-gauge stainless steel needle with blunt tip (Becton Dickinson, Franklin Lakes, N.J.). The positive electrode of the power supply was clamped directly onto the needle. A 3×3 inch copper plate covered with aluminum foil was grounded and used to collect the electrospun fibers. In electrospinning PSBMA, the distance between the needle tip and the collector was set to 21 cm. A 25 kV DC voltage was applied between the needle and the grounded collector. The PSBMA solution was dispensed at a flow rate of 17 μL/min and electrospun at ˜10° C. and 10% relative humidity (RH). The as-spun PSBMA membranes were photo-crosslinked by irradiation under UV light (365 nm, 8W) at 10% RH and a light-to-sample distance of 3 cm for 15 minutes. The samples were then dried for 24 hours at room temperature and 10% RH to remove any residual solvent.

For the PCL electrospun samples, a 9.6% (w/v) PCL solution was prepared in a mixed solvent of methanol and chloroform (1:3, v/v), and electrospun with a flow rate of 10 μL/min and a tip-to-collector distance of 35 cm.

### Scanning Electron Microscopy (SEM):

Surface morphology of the electrospun samples was characterized using an FEI Quanta 200 scanning electron microscope. Dried electrospun membranes were punched into 9 mm-diameter disks and affixed onto aluminum stubs using double-sided adhesive conductive carbon tape. Prior to imaging, sample disks were coated with a thin layer of silver using a K575X Turbo Sputter Coater (Emitech, United Kingdom) at 30 mA for 45 s. Images were captured under high vacuum conditions at 25 kV. With the image analysis program, AxioVs40 (Carl Zeiss Imaging solutions, Germany), a total of 50 well-resolved fibers in the SEM images were analyzed to determine the mean and standard deviation of the fiber diameters.

### Water Absorption:

Water absorption of the PSBMA electrospun membranes was assessed by comparing weights of the dry and hydrated samples. Dry electrospun PSBMA membranes were weighed first and then soaked in water. After 24 h, the hydrated samples were withdrawn from water and the excess surface water was removed with Kimwipe. The samples were weighed again. Water absorption was then calculated according to the following equation: Water absorption (%)=[(Wₛ−W_{d}) /W_{d}]×100%, where Wₛ represents the weight of the membrane after water uptake, and W_{d} is the initial weight of the dry membrane. To test the reversibility of water absorption, the hydrated PSBMA membranes were vacuum-dried at 50° C. for 12 h, weighed, rehydrated for 24 h, and weighed again, followed by the determination of the water absorption percentage.

Water absorption of PSBMA hydrogel was also measured for comparison. To prepare the PSBMA hydrogel, SBS (3.1%, w/v) and APS (8.5%, w/v) were dissolved in 1 mL water, which was then mixed with 1 ml ethanol and 3 ml ethylene glycol, followed by the addition of TEGDMA (264 μL) and SBMA (3.75 g). A mold was built by placing a 0.0381-cm thick Teflon spacer between two clean glass slides. The mixed solution was poured into the mold where it polymerized overnight. After polymerization, the gel was released from the mold and immersed in water, with frequent water changes, to remove the leachates such as unreacted initiators, monomers, and oligomers.

### Protein Adsorption by Enzyme-Linked Immunosorbent Assay (ELISA):

Direct ELISA was used to measure fibrinogen (Fg) adsorption onto the electrospun PSBMA membranes. The electrospun PSBMA membranes were first immersed in water for 24 hours to remove the unreacted initiators, monomers, and oligomers, punched into 9-mm circular disks, dried, and weighed. The sample disks were then placed in individual wells of a 24-well plate in triplicates, washed with 500 μL of PBS, and incubated with 500 μL of 1 mg/mL Fg in PBS at 37° C. for 90 min. To block the areas unoccupied by Fg, the disks were rinsed with 500 μL PBS 5 times and placed in 500 μL of 1 mg/mL BSA and incubated at 37° C. for 90 min. After rinsing with PBS 5 times, the samples were placed in 500 μL of 5.5 μg/mL HRP-conjugated anti-Fg and incubated at 37° C. for 90 min, followed by washing with PBS 5 times. For the color development, the samples were transferred to new wells and 500 μL of 0.1M phosphate-citrate buffer containing 1 mg/mL OPD and 0.03% hydrogen peroxide was added to each well and incubated at 37° C. for 20 min. The reaction was finally stopped by adding 500 μL of 1M sulfuric acid to each well. The absorbance of light at 490 nm was measured by a Tecan Infinite M200 microplate reader (Switzerland). The positive control for this experiment was the PCL electrospun membrane and the negative controls were the PSBMA electrospun membranes with no Fg or anti-Fg added during the ELISA test. The optical density measured at 490 nm was normalized to the polymer mass. The mass-normalized absorbance from PCL electrospun membranes was set to 100% for calculating the relative protein adsorption of other samples.

### Cell Culture:

Endothelial cell is a typical cell type found in wound tissues as well as involved in vascularization of a healing wound. Bovine aortic endothelial cell (BAEC), a commonly used model cell type for studying cell adhesion or resistance on materials, is used in our work to evaluate the resistance of electrospun PSBMA membranes to cell adhesion.

BAECs were maintained in continuous growth on tissue culture polystyrene flasks in Dulbecco's modified Eagle medium (DMEM) supplemented with 10% fetal bovine serum (FBS), 2% penicillin-treptomycin, 1% sodium pyruvate, and 1% nonessential amino acids at 37° C. in a humidified atmosphere containing 5% CO₂. The cells were passaged once a week and discarded after 15 passages.

The electrospun PSBMA membranes and control samples, all with diameter of 9 mm, were soaked in water for 24 hours, sterilized with UV, and rinsed with 70% ethanol and sterile PBS prior to cell seeding. The control materials for this experiment include electrospun PCL, tissue culture polystyrene (TCPS), and PSBMA hydrogel. Confluent cells were detached from the flask surface with trypsin/ethylenediaminetetraacetic acid (0.05%/0.53 mM), suspended in PBS, centrifuged, and diluted in the supplemented medium at a final concentration of 10⁵ cells/mL. One milliliter of cell suspension was then added to each sample and incubated for 48, 72, or 96 hours at 37° C. Cell attachment was then quantified using a Vybrant® MTT cell proliferation assay kit following the manufacturer's protocol. Absorbance of the colored solutions was measured at 540 nm using a Tecan Infinite M200 microplate reader. The electrospun PSBMA and PSBMA hydrogel disks with no cells seeded (i.e., samples are incubated with culture medium only) were also included as control. Statistical analysis were performed using the Student's t-test with p<0.05 considered significant. Data are presented as mean ±standard deviation. To image cells on the electrospun membranes, another set of electrospun PSBMA and PCL samples were cultured with cells following the same procedure. At the end, samples were washed with PBS, stained with 1 mg/ml 4′6-diamidino-2-phenylindole dihydrochloride (DAPI) at 37° C. for 10 minutes, and washed with water prior to imaging. The florescence images were taken on an Olympus BX60 microscope equipped with an Olympus DP71 digital camera.

### Bacterial Adhesion and Zone of Inhibition:

Two bacterial species, Gram-negative P. aeruginosa with a GFP expressing plasmid and Gram-positive S. epidermidis, were used to study bacterial adhesion as well as zone of inhibition. The electrospun PSBMA membranes and control samples, all with diameter of 9 mm, were soaked in water for 24 hours, sterilized with UV, washed with 70% ethanol three times, and rinsed with sterile water three times for further bacterial studies. The control materials used for the bacterial adhesion studies were electrospun PCL, TCPS, glass, and PSBMA hydrogel.

P. aeruginosa and S. epidermidis were first cultured on separate agar plates overnight at 37° C. in lysogeny broth (LB) (BD, Franklin Lakes, N.J.). Several colonies of each bacterium were used to inoculate 5 ml of LB medium separately. The initial cultures were incubated at 37° C. under shaking at 250 rpm for 18 h and then diluted to an optical density of 0.1 at 600 nm. Two milliliters of bacterial solution was then added into each well containing the PSBMA electrospun membrane or control and incubated at 37° C. For the 3 h study, samples were washed with sterile water 3 times after 3 hours of incubation and microscope images were taken. For the 24 h study, after 3 hours of incubation at 37° C., the samples were washed with sterile water 3 times to remove planktonic bacteria and then transferred to new wells. Two milliliters of fresh LB medium was then added into each well and incubated at 37° C. for another 24 hours. Samples were finally washed with sterile water three times and imaged. Samples incubated with P. aeruginosa were directly imaged by Zeiss Meta 510 confocal laser scanning microscope equipped with a helium-neon laser. Samples incubated with S. epidermidis were stained with Live/Dead BacLight kit prior to imaging. All samples were tested in triplicates.

The zone of inhibition study was carried out to test the antimicrobial activity of the silver-impregnated electrospun PSBMA membranes. The bacterial lawns were prepared by inoculation of agar plates with P. aeruginosa or S. epidermidis and incubation at 37° C. overnight. The electrospun PSBMA membranes were loaded with silver ions by immersion in an aqueous solution of silver nitrate (5.7%, w/v) for 5 hours. Electrospun PSBMA and electrospun PCL membranes were used as controls. Using a modified Kirby Bauer technique, the samples and the controls were placed on the bacterial lawns and incubated at 37° C. for 24 hours. The zone of inhibition was then measured around the electrospun membranes. The amount of silver impregnated into the dressing was determined by comparing Ag⁺concentrations in AgNO₃ solution before and after soaking the dressing in AgNO₃ solution. The silver concentration was determined using a 710-ES series inductively coupled plasma—optical emission spectrometer (Agilent Technologies, Santa Clara, Calif.).

### Results and Discussion

### Electrospinning of Water-Stable PSBMA:

PSBMA was electrospun in this work. Fibrous membrane produced by the electrospinning process has high surface-area-to-volume ratio, porous structure, and conformability, as compared to hydrogel.

To use electrospun membranes as wound dressings, the membranes need to be stable (i.e., remain integral) in the aqueous environment. Previous research showed that fibrous membranes electrospun from solutions of pure PSBMA were soluble in water even when the membranes were fabricated from a high molecular weight polymer. Superhydrophilicity of PSBMA suggests a stronger interaction between polymer chains and solvent molecules (i.e., H₂O) compared to the polymer chain-chain interaction. Chemically crosslinked PSBMA fibers are thus needed for the wound dressing applications. Hydrogel (i.e., crosslinked network) of PSBMA is water stable; however hydrogel formulation and experimental conditions cannot be directly applied to the electrospinning since electrospinning requires a viscous solution with a relatively consistent viscosity throughout the process. In preparing hydrogels of PSBMA following the literature procedure, it was observed that there was a rapid increase in the reactant solution viscosity prior to the gel formation, meaning that there was only a very short time window where the solution can be electrospun. Once hydrogel is formed, conversion of such hydrogel into fibrous form is impossible since the cross-linked bulk gel can neither dissolve nor melt.

A three step polymerization-electrospinning-photocrosslinking process was developed to fabricate the water-stable crosslinked electrospun PSBMA membrane. Mixture of the monomer, crosslinker, redox initiators, and photoinitiator was first reacted via free radical polymerization to form a precursor solution to achieve a viscosity necessary for electrospinning The reaction was then cold-quenched to stop further increase in solution viscosity. Next, the viscous precursor solution was electrospun at a low temperature. The actions of quenching the polymer solution and keeping the electrospinning solution at a low temperature both assure a relatively consistent solution viscosity during the electrospinning and prevent the gel formation which otherwise would clog the needle. Upon completion of electrospinning, the membrane was white, pliable and stretchable. A UV treatment followed the electrospinning process immediately for further polymerization and crosslinking. With the quenched reaction (i.e., incomplete polymerization), double bonds were available from the unreacted monomer molecules or on the polymer chains, which can be utilized for further polymerization. The addition of TEGDMA further enhanced the availability of double bonds, leading to the crosslinked PSBMA with the aid of the UV initiator, Irgacure 2959. Before the UV treatment, the electrospun PSBMA membrane dissolved readily in water. The UV-treated membrane was water - stable and more rigid, an indication that the covalent crosslinking had taken place within fibers as well as among fibers. Comparison of the physical dimensions of the membranes showed no size differences before and after the UV treatment. Due to the superhydrophilicity of PSBMA, relative humidity (RH) of the environment where the polymer jet turns into fibers strongly affects the formation and morphology of the PSBMA fibers. A 10% RH was used in this work to obtain fibrous electrospun membranes. The humidity effect on formation of electrospun fibers has been observed for other polymers such as polyethylene oxide and poly(vinylpyrrolidone).

### Scanning Electron Microscopy:

The electrospun membranes appear white and ridged when dry, semi-transparent white and pliable when hydrated. SEM images show that the electrospun PSBMA membranes possess a porous structure with randomly oriented nonwoven fibers, with an average diameter of 1110.4±145.8 nm. Fibers appear smooth with fusion of the overlapping fibers where they are joined. Since Irgacure 2959 is available within the fibers as well as on the surface of the fibers, it is possible that once the UV initiator is activated crosslinking would take place within fibers and among fibers.

### Water Absorption:

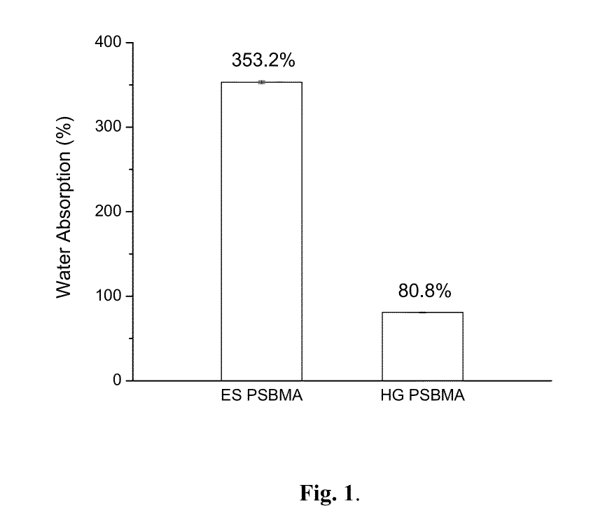

The PSBMA fiber is superhydrophilic. Water drops were drawn immediately into the fibrous membranes when they came in contact with the membrane surfaces. To characterize the hydration capacity of the electrospun PSBMA membranes, the water absorption level was determined based on the weight difference of the membranes before and after water uptake relative to their dry mass. The electrospun membranes of PSBMA exhibited average water absorption of 353.2%, more than 4 times compared to the PSBMA hydrogels which only absorbed 80.8% water (FIG. 1). The hydrated electrospun membranes were then dried and tested for the water absorption again. A water absorption percentage of 354% was obtained, showing excellent reversibility of water absorption of the electrospun PSBMA membranes. Factors affecting water absorption include water molecules bound to the fiber surfaces, water molecules held between polymer chains, as well as those trapped in the porous structure of the membrane. High surface-area-to-volume ratio of the electrospun PSBMA fiber led to more water bound on the fiber surface via ionic solvation. Water was also held between polymer chains and in the pores. All these factors contribute to the much higher water absorption of the electrospun PSBMA membrane compared to the hydrogel. The superior water absorption of the electrospun PSBMA membrane makes it a great choice as a wound dressing material to remove fluid from highly exudating wounds and aid in resisting undesirable biofouling.

### Protein Resistance of Electrospun PSBMA:

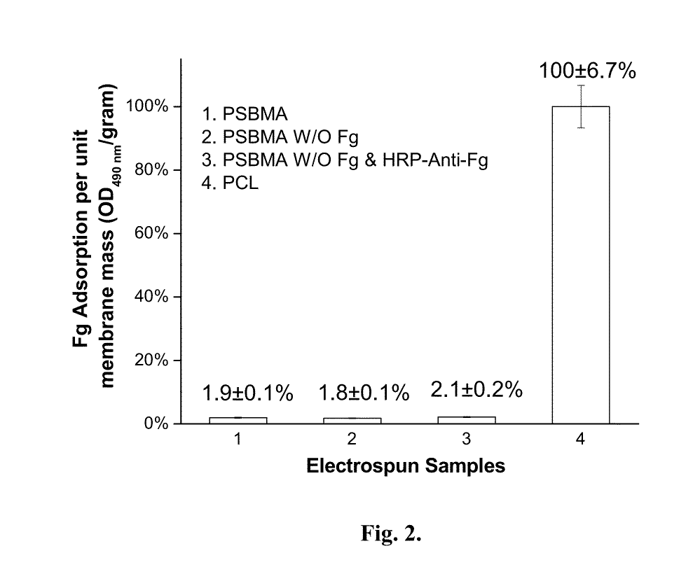

Enzyme-linked immunosorbent assay (ELISA) was carried out to assess protein resistance of the electrospun PSBMA membranes. Fibrinogen (Fg), a typical protein used in evaluation of nonfouling properties of biomaterials, was used here as the model protein. Fg can easily adsorb to a wide range of material surfaces and it is a coagulation protein involved in platelet aggregation and blot clot formation. Two sets of electrospun PSBMA samples, one without Fg applied during ELISA, the other without both Fg and anti-Fg added during ELISA, were included as the negative controls. Electrospun PCL was used as the positive control since it promotes protein adsorption and cell attachment. The absorbance of all samples was normalized to their mass to eliminate the impact of mass difference among samples. The mass-normalized absorbance of the PCL control at 490 nm was set to 100%. FIG. 2 shows that protein adsorption on the electrospun PSBMA samples was significantly suppressed compared to the PCL control. Even though all PSBMA samples show absorbance of about 2% relative to PCL, it is not an indication of Fg adsorption on the PSBMA electrospun membranes. Statistical analysis shows that the protein adsorption on electrospun PSBMA is not significantly different from two negative controls, attesting to the complete protein resistance of PSBMA electrospun membranes. As discussed above, PSBMA electrospun membranes possess a high degree of water absorption capacity. Strong hydration, arising from electrostatic attractions between the charges on the pendant groups and water molecules, contributes to the protein resistance of the fibrous PSBMA membranes.

### Cell Resistance of Electrospun PSBMA:

In a comparison of microscope images of cells attached to electrospun membranes of PSBMA and PCL, it was evident that there was no cell attachment on the electrospun PSBMA membranes after culturing with BAECs for 48, 72, or 96 hours. In contrast, the progression of cell attachment on PCL was clear. The PCL samples showed some cell attachment along the fibers at 48 hours and more at 72 hours. At 96 hours, a very high density of cells was observed attaching and covering most of the PCL fibers. This is consistent with the ELISA results that surfaces of electrospun PSBMA membranes are resistant to protein adsorption. Cells could not attach to the surfaces since the surfaces were devoid of any adhesive proteins either from the FBS-supplemented medium or secreted by the cells. PCL on the other hand exhibits a high level of protein adsorption (FIG. 2), providing an ideal environment for cell attachment and proliferation.

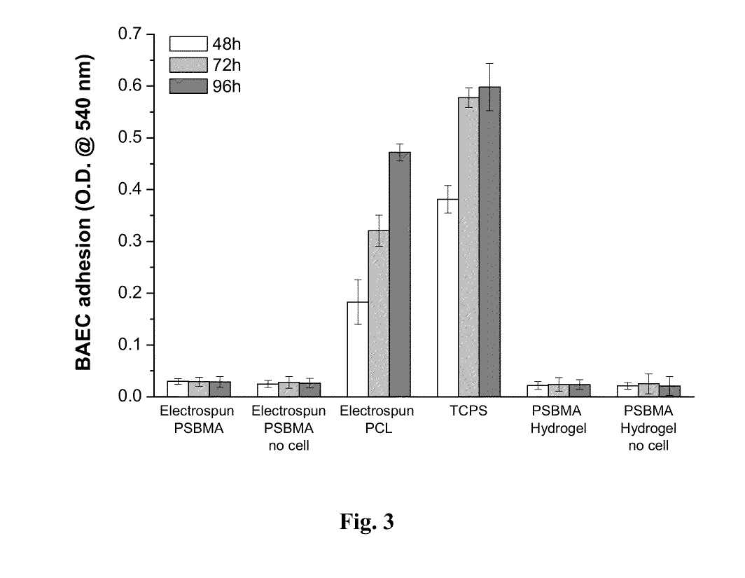

Cell adhesion was further quantified using MTT cell proliferation assay. FIG. 3 shows that the electrospun PSBMA membranes exhibited much lower optical density compared to the electrospun PCL membranes and TCPS substrates, both of which promoted cell adhesion and proliferation significantly. The electrospun PSBMA membranes cultured with medium only (i.e., zero cell seeding) under otherwise identical conditions, was used as negative control. Statistical analysis shows the cell attachment on electrospun PSBMA membranes is not significantly different from the negative control. Similar results are obtained for PSBMA hydrogel samples with or without seeded cells. Both microscope images and MTT results demonstrate that the PSBMA electrospun membranes are resistant to cell attachment.

The unique properties that cells and proteins do not adhere to the electrospun PSBMA membrane will lead to multiple benefits for wound care, if such material is used as wound dressing. Firstly, such dressing will cause no patients' pain in situ and upon removal of the dressing. Secondly, when the wound dressing is removed, the newly formed skin layer will not be disturbed. Thus there will be less chance of creating another wound, leading to quicker healing. This type of nonadherent wound dressing is extremely important for the care of patients with large areas of wounds such as severely burned patients, or for the care of donor site wounds. Pain and discomfort have been reported to occur more frequently from donor site wounds than at the recipient sites.

### Bacterial Resistance of Electrospun PSBMA:

Adhesion of microorganisms onto synthetic surfaces, a necessary step before colonization and biofilm formation, is a prerequisite in initiation of infection. To combat biofilm formation which can potentially affect most medical devices including wound dressings, it is crucial to stop or reduce the initial bacterial adhesion onto these surfaces. Physicochemical properties of surfaces of both biomaterials and bacteria dictate the bacterial adhesion to biomaterials. Hydrophilic uncharged surfaces, such as poly(ethylene oxide) and PSBMA, have shown great resistance to bacterial attachment. Bacterial adhesion to synthetic surfaces is also affected by presence or absence of adhesive proteins. For example, Herrmann and coworkers observed an increase in the bacterial adhesion when polymer surfaces were coated with proteins such as fibrinogen and fibronectin.

Typically, bacterial strains used in biocompatibility studies are chosen based on their relevance and prevalence to applications of the materials as well as their ability to form biofilm. Other factors taken into account are to include bacteria with different cell envelopes and those having different attachment mechanisms. Here, P. aeruginosa, Gram-negative bacteria, with a thin cell wall and an outer phospholipid bilayer, and S. epidermidis, Gram-positive bacteria, were selected for our study. Both species are present in large numbers at the wound sites and play an important role in dermal wound infections. S. epidermidis tends to infect biomedical implants and transcutaneous devices.

After a 3-hour bacterial incubation, it was clear that the PSBMA electrospun membrane and hydrogel exhibited very little bacterial attachment for both P. aeruginosa and S. epidermidis in comparison to the electrospun PCL, TCPS, and glass. Bacterial adhesion at 24 hours showed that the PSBMA electrospun membranes still exhibited the lowest bacterial adhesion for both species. It has to be noted that the electrospun samples inherently have rougher surface morphology compared to the flat TCPS and glass substrates; in addition, electrospun membranes have pores which may trap bacteria on surfaces and within the membranes. Considering that, still electrospun PSBMA membranes show the lowest bacterial adhesion when compared to all other samples. Resistance of the electrospun PSBMA membranes to bacterial attachment corroborates the high hydration capacity, resistance to protein adsorption, and resistance to cell adhesion of the membranes. Bacterial resistance of electrospun PSBMA makes it a promising material for wound dressing which can prevent attachment and entry of the environmental pathogens to the wound. The dressing applied to the wound also does not need to be replaced as often as the case for a fouling dressing, which leaves less chance of introducing new bacteria with repeated exposure of the wound site to the environment and causes less pain for the patients.

### Zone of Inhibition of Ag+—Incorporated Electrospun PSBMA:

Ionic silver is a well-known broad-spectrum antimicrobial agent, effective against a variety of wound pathogens including antibiotic-resistant bacteria. Microorganisms with resistance to the antimicrobial activity of Ag are very rare and silver has been used in burn care for more than 50 years.

Here AgNO₃ was incorporated into the electrospun PSBMA membrane through ionic interactions. PSBMA contains a large amount of anionic So₃⁻ groups (one SO₃⁻ per pendant group), allowing for the incorporation of cationic silver ions with high drug loading. The antimicrobial activity of the Ag⁺⁻ impregnated membrane was then determined using a zone-of-inhibition method. This method mimics the clinical use of wound dressing and predicts its microbicidal activity at the dressing-wound interface.

After the disks of PSBMA electrospun membrane were immersed into the silver nitrate aqueous solution, diameter of the samples was found increasing due to shielding of charges on the polymer molecules by silver and nitrate ions, an indication of successful incorporation of antimicrobial silver into the membrane. The amount of silver impregnated into the electrospun PSBMA membrane was determined to be 0.14 g Ag/g membrane. The electrospun PSBMA membranes infused with silver nitrate were found to inhibit growth of both P. aeruginosa and S. epidermidis. Zone of inhibition was 6.3 mm for P. aeruginosa and 3.6 mm for S. epidermidis after 24 hours of incubation. The uniform diameter of the zone of inhibition is an indication of homogeneous impregnation of silver nitrate into the PSBMA electrospun membranes as well as uniform release of silver over the bacterial incubation time.

### CONCLUSIONS

Water-stable porous membrane of electrospun PSBMA hydrofibers was successfully developed using a polymerization-electrospinning-photocrosslinking process. The membrane exhibits superior water absorption and resists protein adsorption, cell attachment, and bacterial adhesion. Anti-microbial activities of the Agtimpregnated electrospun PSBMA membrane was shown against both Gram-positive S. epidermidis and Gram-negative P. aeruginosa.

Such electrospun PSBMA membrane is ideal for a novel type of nonadherent and superabsorbent wound dressings, which can effectively manage exudates, support wound healing by maintaining moist wound environment, eliminate patients' pain in situ and on dressing removal, avoid formation of new wounds upon dressing removal, prevent attachment and entry of environmental bacteria, offer broad-spectrum antimicrobial activity, and allow gas exchange.

Various modifications and alterations that do not depart from the scope and spirit of this invention will become apparent to those skilled in the art. This invention is not to be duly limited to the illustrative embodiments set forth herein.

In light of the foregoing, it should be appreciated that the present invention significantly advances the art by providing nonadherent and superabsorbent wound dressings and methods of making. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A non-adhesive, water-absorbent wound dressing comprised of polymerized zwitterionic monomers according to the following structure: 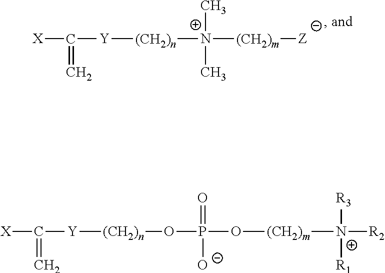
wherein X is selected from the group consisting of hydrogen or a methyl group; Y is selected from the group consisting of esters and amides; n is an integer from 1 to 4, m is an integer from 1 to 5, Z is selected from the group consisting of SO₃ or COO; and R₁, R₂ and R₃ are each any moiety.

2. The wound dressing of claim 1, wherein X is methyl, Y is ester, n is 2, m is 3, and Z is SO₃, such that the monomer is sulfobetaine methacrylate (SBMA).

3. The wound dressing of claim 1, X is methyl, Y is ester, n is 2, m is 2, and Z is COO, such that the monomer is carboxybetaine methacrylate (CBMA).

4. The wound dressing of claim 1, X is hydrogen, Y is amide, n is 2, m is 1, 3, or 5, and Z is COO, such that the monomer is carboxybetaine acrylamide (CBAA).

5. The wound dressing of claim 1, X is methyl, Y is ester, n is 2, m is 2 and R₁, R₂ and R₃ are each methyl groups, such that the monomer is poly(phosphoryl)choline.

6. The wound dressing of claim 1, the polymerized monomers form a non-woven electrospun fabric.

7. The wound dressing of claim 1, where the wound dressing further includes an antimicrobial cations associate with negatively charged species of the monomers.

8. A method of creating a non-adhesive, water-absorbent wound dressing comprising:
creating a mixture of monomers, crosslinker, initiator, and solvent, the monomer being selected from the following structures: 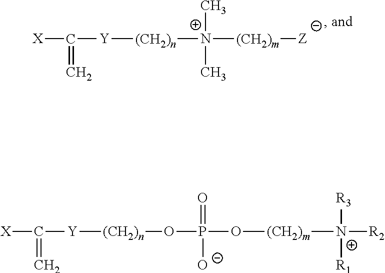
wherein X is selected from the group consisting of hydrogen or a methyl group; Y is selected from the group consisting of esters and amides; n is an integer from 1 to 4, m is an integer from 1 to 5, Z is selected from the group consisting of SO₃ or COO; and R₁, R₂ and R₃ are each any moiety;
initiating polymerization of the monomers to begin polymerization of the monomers;
quenching the polymerization of the monomers when the mixture has a viscosity suitable for electrospinning;
electrospinning the polymer mixture to form a fibrous membrane; and
thereafter further polymerizing and crosslinking the fibrous membrane.

9. The method of claim 8, wherein said step of quenching involves a reduction in the temperature of the mixture, thus at least slowing the rate of polymerization.

10. The method of claim 8, wherein said step of crosslinking also includes further polymerization.

11. The method of claim 8, wherein, in said step of electrospinning, the humidity to which the polymer mixture is exposed is controlled.

12. The method of claim 11, wherein the humidity is controlled to be less than 15%.

13. The method of claim 8, further comprising adding an antimicrobial cation to the fibrous membrane following the UV crosslinking step.

14. The method of claim 13, wherein said step of adding and antimicrobial cation includes immersing the fibrous membrane in a solution containing antimicrobial cations that associate with local negative charges on the polymers forming the fibrous membrane.
